(19) United States
(12) Patent Application Publication
(10) Pub. No.: US 2023/0142353 A1
(43) Pub. Date: May 11, 2023

(54) IMPLANT DEVICES AND METHODS FOR TREATMENT OF CERVICAL CANCER

(71) Applicant: Washington University, St. Louis, MD (US)

(72) Inventors: Abdel Kareem Azab, St. Louis, MO (US); Cinzia Federico, St. Louis, MO (US); Jennifer Sun, St. Louis, MO (US); Julie Schwarz, St. Louis, MO (US)

(73) Assignee: Washington University, St. Louis, MO (US)

(21) Appl. No.: 17/916,180

(22) PCT Filed: Mar. 30, 2021
(86) PCT No.: PCT/US21/24978

§ 371 Date: Sep. 30, 2022

Related U.S. Application Data
(60) Provisional application No. 63/001,987, filed on Mar. 30, 2020.

(51) Int. Cl. A61K 9/00; A61K 49/00; A61P 35/00; A61P 15/00; A61K 47/10; A61K 33/243

(52) U.S. Cl. CPC A61K 9/0034; A61K 49/006; A61P 35/00; A61P 15/00; A61K 49/0032; A61K 47/10; A61K 33/243

(57) ABSTRACT

A method of treating cervical cancer in a patient in need is described that includes implanting a fast release implant containing an effective amount of Cis-Pt within a cervical cancer lesion. The implant includes a polymer and a therapeutic load homogenously distributed throughout the polymer. The implant assumes a solid phase at room temperature and assumes a liquid phase at a body temperature of the patient.

### CROSS-REFERENCE TO RELATED APPLICATIONS

This application claims the benefit of priority to U.S. Provisional Application No. 63/001,987 filed on Mar. 30, 2020, the content of which is incorporated by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to compositions and methods of treatment of cervical cancer.

### BACKGROUND OF THE DISCLOSURE

Cervical cancer represents the fourth most frequent malignancy in the world among women. Despite encouraging improvements in screening and prevention, cervical cancer mortality has remained stable for the past four decades.

Current treatment options for cervical cancer include radical hysterectomy or definitive chemo-radiation depending primarily on the clinical stage of the disease. The current standard-of-care radiation therapy for locally advanced cervical cancer consists of conventionally fractionated radiation to the pelvis (once daily) in addition to hypofractionated boost dose delivered directly to the tumor via intracavitary brachytherapy. Systemic cisplatin-based chemotherapy is concurrently administered with radiation, which has been shown to improve local control as well as overall survival outcomes compared to radiation alone in randomized controlled clinical trials. However, cisplatin induces serious dose-limiting systemic toxicities.

In addition, systemic delivery of treatments for cervical cancer may not be the most suitable administration method for all patients, depending on the stage of the cervical cancer. Systemic delivery systems for chemotherapy (such as nanoparticles) have shown promising results for the treatment of metastatic cervical cancer due to the disseminated nature of the disease. However, more than 80% of cervical cancer cases are diagnosed at local or regional stages. Therefore, a systemic approach may not be the ideal strategy for the treatment of local and regional cervix cancer cases.

Cisplatin (Cis-Pt) is an alkylating antineoplastic agent used to treat several malignancies including cervical cancer. Despite Cis-Pt's potent anticancer activity, it possesses serious dose-limiting side effects such as nausea and vomiting, kidney damage, neuropathy, hearing loss, and bone marrow suppression. These dose-limiting side effects can negatively affect the clinical efficacy of Cis-Pt, causing chemotherapy noncompliance and discontinuation as potential drivers for inefficacious therapy and disease relapse. The current standard-of-care Cis-Pt dosage for cervical cancer treatment is once-weekly intravenous (IV) injection of 40 to 70 mg/m²; however, previous studies have shown that 24 hours after IV injection of a 100 mg/m² dose resulted in intratumoral platinum (Pt) concentration of only 0.9 ng/mg. This concentration is far below the previously reported Pt amounts needed to achieve cytotoxic effects in cervical cancer cells in vitro. The development of a new drug delivery system designed to maximize the intratumor accumulation of Cis-Pt while minimizing the dose-limiting side effects has the potential to dramatically improve therapeutic outcomes.

### SUMMARY OF THE DISCLOSURE

In one aspect, a fast release implant for the treatment of a cervical cancer is disclosed that includes a polymer and a therapeutic load homogenously distributed throughout the polymer. The implant assumes a solid phase at room temperature and assumes a liquid phase at a body temperature of a patient. In some aspects, the polymer includes a mixture of a high MW PEG and a low MW PEG. In some aspects, the high MW PEG is PEG3350 and the low MW PEG is PEG400. In some aspects, the implant includes molar ratio of high MW PEG:low MW PEG of 80:20. In some aspects, the therapeutic load includes Cis-Pt. In some aspects, the therapeutic load includes Cis-Pt at a concentration of about 2 mg Cis-Pt/g PEG. In some aspects, the therapeutic load further includes a dye or a contrast agent. In some aspects, the dye or the contrast agent is selected from the group consisting of trypan blue, DiR, and any combination thereof.

In another aspect, a method of treating cervical cancer within a patient in need is disclosed that includes implanting a fast release implant containing an effective amount of Cis-Pt within a cervical cancer lesion. The implant includes a polymer and a therapeutic load homogenously distributed throughout the polymer. The implant assumes a solid phase at room temperature and assumes a liquid phase at a body temperature of a patient. In some aspects, the polymer includes a mixture of a high MW PEG and a low MW PEG. In some aspects, the high MW PEG is PEG3350 and the low MW PEG is PEG400. In some aspects, the implant includes a molar ratio of high MW PEG:low MW PEG of 80:20. In some aspects, the therapeutic load includes a therapeutically effective amount of Cis-Pt. In some aspects, the therapeutic load includes Cis-Pt at a concentration of about 2 mg Cis-Pt/g PEG. In some aspects, the therapeutic load further includes a dye or a contrast agent. In some aspects, the dye or the contrast agent is selected from the group consisting of trypan blue, DiR, and any combination thereof.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a graph summarizing the concentration of platinum (Pt) in patient biopsy samples (n=20) and in cell lines treated at Av-IC₅₀ (n=5) as measured by inductively coupled plasma mass spectrometry (ICP-MS).

FIG. 1B is a bar graph summarizing the relative number of surviving clones as a percent of untreated clones at different Cis-Pt and radiation doses; p values represent comparisons against 0 μM Cis-Pt condition at corresponding radiation doses (* denotes P<0.01).

FIG. 2A is a graph summarizing the in vitro dissolution profile of empty polyethylene glycol (PEG) implants as a percent of the initial size.

FIG. 2B is a graph summarizing the in vitro drug release profile from the implants of FIG. 2A loaded with intracellular cisplatin (Cis-Pt), calculated as percent of the total dose.

FIG. 2C contains a series of images showing the in vivo dissolution of the DiR-loaded implants of FIG. 2B in mice (n=5).

FIG. 2D is a graph summarizing the in vivo dissolution profile of the implants of FIG. 2C, calculated as percent area in the abdomen with DiR signal.

FIG. 3A is a series of images schematically illustrating the implantation of a cisplatin (Cis-Pt) delivery device in an in vivo subcutaneous tumor model.

FIG. 3B is a bar graph summarizing the in vivo biodistribution profile of Cis-Pt (n=5) within the subcutaneous tumor model of FIG. 3A, measured by platinum (Pt) levels in various organs, 24 hours after Cis-Pt-Implant or Cis-Pt-IV administration. (*P<0.05; 1 †P<0.01; ‡P<0.001); insert on top right shows a zoomed-in view of the biodistribution profile.

FIG. 4A is a diagram illustrating the assessment of platinum (Pt) distribution in different sections of the tumors after treatment with empty or Cis-Pt implants using 2 different methods: inductively coupled plasma mass spectrometry (ICP-MS) and immunofluorescence (IF).

FIG. 4B is a graph summarizing Pt concentrations in each tumor layer from the Cis-Pt implant group (n=3) of FIG. 4A measured by ICP-MS analysis and adjusted with background signal from the empty implant group (n=3).

FIG. 4C contains fluorescence microscopy images of regions of tumor sections, stained with antibody for Cis-Pt-induced DNA adducts (AF488) and counterstained with DAPI; gray lines indicate edges of tissues.

FIG. 5A is a graph summarizing tumor progression for treatment conditions Vehicle-IV, Cis-Pt-IV, or Cis-Pt-Implant, without irradiation.

FIG. 5B is a graph summarizing tumor progression for treatment conditions Vehicle-IV, Cis-Pt-IV, or Cis-Pt-Implant, with 2 Gy/d irradiation for 5 consecutive days.

FIG. 5C is a graph summarizing tumor progression for Cis-Pt-Implant with fractioned radiation (2 Gy/d×5 days) or high-dose brachytherapy (8 Gy once); *P<0.05; †P<0.01; ‡P<0.001.

FIG. 6 is a graph summarizing cell proliferation in various cervix cancer cell lines treated with various concentrations of Cis-Pt; IC₅₀ of an average response of all cancer cell lines is denoted by dashed lines.

FIG. 7 is a graph summarizing Pt levels obtained from patient tumor biopsies obtained over a range of days after a Cis-Pt treatment administered at a dose of 6 μM, corresponding to the IC₅₀ level of FIG. 6.

FIG. 8A contains a series of bar graphs summarizing cell proliferation of various cervix cancer cell lines over a period of 48 hours following radiation therapy accompanied by administration of Cis-Pt at a clinically relevant dose of 1 μM.

FIG. 8B contains a series of bar graphs summarizing cell proliferation of various cervix cancer cell lines over a period of 48 hours following radiation therapy accompanied by administration of Cis-Pt at a dose of 6 μM, corresponding to the IC₅₀ level of FIG. 6.

FIG. 9A is a graph summarizing changes in absolute tumor volumes over 10 days of Cis-Pt treatment by injection or by release from an implant.

FIG. 9B is a graph summarizing changes in absolute tumor volumes over 10 days of irradiation at 2 Gy/day combined with Cis-Pt treatment by injection and by release from an implant.

FIG. 9C is a graph summarizing changes in absolute tumor volumes over 7 days of Cis-Pt treatment by release from an implant accompanied by irradiation administered in 5 treatments of 2 Gy or a single treatment of 8 Gy.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE INVENTION

In various aspects, devices and methods of treatment for cervical cancer are disclosed. In some aspects, a drug delivery system is described that may be implanted into the cervical cavity to release cisplatin (Cis-Pt) into the depth of the tumor tissue associated with the cervical cancer. The drug delivery system is designed to promote the fast-release of the Cis-Pt into the tumor tissue to circumvent clearance by the physiological secretions of the vaginal tract. Since most diagnosed cervical cancers are local and regional, the localized therapy provided by the disclosed drug delivery system could supplement existing treatments.

In some aspects, the current standard-of-care brachytherapy treatments (systemic cisplatin-based chemotherapy concurrently administered with radiation) may be modified to incorporate the administration of Cis-Pt using the disclosed drug delivery system. The disclosed drug delivery system may be implanted into the cervix and release its load within the timeframe of the radiotherapy treatment and act as radio-sensitizing adjuvant/neoadjuvant therapy.

The current standard-of-care systemic (injection) administration of cisplatin provides a subtherapeutic dose. Without being limited to any particular theory, localized delivery of chemotherapy in various solid tumors is thought to improve therapeutic efficacy in the tumor and to limit systemic toxicities. The localized treatment approach in cervical cancer provided by the disclosed drug delivery system improves local tumor control and reduces side effects. The cervix is accessible for intra-vaginal implantation of local drug delivery devices directly adjacent to the cancer tissue. Existing intra-vaginal devices for delivery of chemotherapy in cervical cancer include vaginal rings, vaginal gels, and cervical patches, but these devices are placed on the external side of the cervix, which in many cases does not promote delivery into the deeper parts of the cervical tumor. In addition, the long-term release properties of these existing intra-vaginal devices are limited by the rapid clearance of the active compounds by the physiological discharge of mucus produced by glands of the cervix.

In various aspects, the disclosed drug delivery system includes a polyethylene glycol (PEG)-based drug delivery implant with fast dissolution properties for localized delivery of Cis-Pt chemotherapy to cervical cancer. These implants will improve Pt accumulation in the cervical tumor and improve therapeutic efficacy in combination with radiation treatment, while simultaneously reducing Pt distribution in other organs and Pt-associated side effects.

As demonstrated in the examples below, the levels of Cis-Pt found in biopsies isolated from cervical cancer patients treated with standard-of-care 40 mg/m² injections were significantly lower than the levels of Pt found in cell lines after treatment with an efficacious concentration in vitro (Av-IC₅₀). These low levels of Pt administered to the cancer patients did not achieve significant killing or radio-sensitivity in vitro (using both clonogenic assay and MTT proliferation assay) or in vivo. These results suggest that the current clinical standard-of-care systemic administration of Cis-Pt provides a subtherapeutic dose for the treatment of cervical cancer.

The biodistribution studies described in the examples below revealed clear advantages for the localized implant as compared to IV injection of the same dose of Cis-Pt. The localized implant demonstrated a drastic 73.4-fold higher accumulation of Cis-Pt in the tumor compared with systemic delivery. Additionally, it resulted in 3.7-fold and 5.26-fold lower accumulation in the blood and kidney, and negligible levels in distant normal organs. Localized Cis-Pt delivery also achieved an 80-fold higher drug content at the tumor site compared with the peripheral blood, whereas systemic delivery exhibited an opposite tumor-to-blood ratio of 0.3. These results demonstrate a remarkable improvement of the specificity of Cis-Pt delivery to the tumor using the disclosed Cis-Pt implant. Localized delivery of Cis-Pt using the disclosed implant resulted in complete inhibition of tumor growth, either alone or in combination with fractionated radiation therapy or brachytherapy. The results described below in the examples demonstrate that localized intracervical Cis-Pt delivery dramatically improves therapeutic outcomes of Cis-Pt-based chemo-radiation therapy in cervical cancer.

In various additional aspects, the localized delivery of active compounds to the cervix using the disclosed implant is suitable for use in the delivery of other potent drugs that are clinically limited by high systemic toxicity, including, but not limited to, DNA damage response inhibitors.

In various aspects, the drug delivery system includes an implant based on polyethylene glycol PEG, an FDA-approved biocompatible polymer used for drug delivery and tissue engineering applications that is highly soluble in an aqueous milieu, such as the cervix. The implant is solid at room temperature to allow insertion into the depth of the cervical cavity. As demonstrated in the Examples below, the implant's dissolution and drug release rates were suitable for a fast release application, as it was able to dissolve and release its cargo within 30 minutes in vitro and in vivo.

Without being limited to any particular theory, the local release of Cis-Pt provided by the PEG-based implants is thought to enhance Pt accumulation in the cervical tumor and to improve therapeutic efficacy in combination with radiation treatment, while simultaneously reducing Pt distribution in other organs and Pt associated side effects.

In various aspects, the fast-release implants are produced using a combination of low and high molecular weights polyethylene glycols (PEGs). The implants are produced by forming a molten mixture of low and high molecular weights PEGS, and homogenously stirring a therapeutic load into the molten polymers mixture. The molten mixture of PEGs and therapeutic load may be formed into implants by pouring the mixture into molds and cooling to solidify the mixture into implants that are solid at room temperature.

In various aspects, any suitable low molecular weight PEG may be used to form the implant including, but not limited to, PEG400. In various other aspects, the low molecular weight PEG may be any PEG with a molecular weight of less than about 1000, less than about 900, less than about 800, less than about 600, less than about 550, less than about 500, less than about 450, less than about 400, less than about 350, less than about 300, less than about 250, less than about 200, less than about 150, or less than about 100. In various additional aspects, the low molecular weight PEG may be any PEG with a molecular weight ranging from about 50 to about 1000, from about 100 to about 750, from about 200 to about 600, from about 300 to about 500, and from about 350 to about 450. In various additional aspects, the low molecular weight PEG may be any PEG with a molecular weight ranging from about 50 to about 150, from about 100 to about 200, from about 150 to about 250, from about 200 to about 300, from about 250 to about 350, from about 300 to about 400, from about 350 to about 450, from about 400 to about 500, from about 450 to about 550, from about 500 to about 600, from about 550 to about 650, from about 600 to about 700, from about 650 to about 750, from about 700 to about 800, from about 750 to about 850, from about 800 to about 900 and from about 850 to about 1000.

In various other aspects, any suitable high molecular weight PEG may be used to form the implant including, but not limited to, PEG3350. In various other aspects, the high molecular weight PEG may be any PEG with a molecular weight of at least about 1000, at least about 2000, at least about 3000, at least about 4000, at least about 5000, at least about 6000, at least about 7000, at least about 8000, at least about 9000, at least about 10000, at least about 15000, at least about 20000, at least about 30000, at least about 40000, at least about 50000, at least about 60000, at least about 70000, at least about 80000, at least about 90000, or at least about 100000. In various additional aspects, the high molecular weight PEG may be any PEG with a molecular weight ranging from about 1000 to about 100000, from about 1500 to about 50000, from about 2000 to about 25000, from about 2500 to about 10000, from about 3000 to about 5000, and from about 3200 to about 4000. In various additional aspects, the low molecular weight PEG may be any PEG with a molecular weight ranging from about 1000 to about 3000, from about 2000 to about 4000, from about 3000 to about 5000, from about 4000 to about 6000, from about 5000 to about 10000, from about 7500 to about 25000, from about 20000 to about 40000, from about 30000 to about 50000, from about 40000 to about 60000, from about 50000 to about 70000, from about 60000 to about 80000, from about 70000 to about 90000, or from about 80000 to about 100000.

In various aspects, the disclosed implants contain high MW PEGs and low MW PEGS at any suitable ratio including, but not limited to, a molar ratio (high MW:low MW) of 80:20. In various additional aspects, the molar ratio is at least 1:100, at least 5:95, at least 10:90, at least 15:85, at least 20:80, at least 25:75, at least 30:70, at least 35:65, at least 40:60, at least 45:55, at least 50:50, at least 55:45, at least 60:40, at least 65:35, at least 70:30, at least 75:25, at least 80:20, at least 85:15, at least 90:10, at least 95:5, or at least 99:1. In various additional aspects, the molar ratio ranges from about 1:99 to about 10:90, from about 5:95 to about 15:85, from about 10:90 to about 20:80, from about 15:85 to about 25:75, from about 20:80 to about 30:70, from about 25:75 to about 35:65, from about 30:70 to about 40:60, from about 35:65 to about 45:55, from about 40:60 to about 50:50, from about 45:55 to about 55:45, from about 50:50 to about 60:40, from about 55:45 to about 65:35, from about 60:40 to about 70:30, from about 65:35 to about 75:25, from about 70:30 to about 80:20, from about 75:25 to about 85:15, from about 80:20 to about 90:10, from about 85:15 to about 95:5, and from about 90:10 to about 99:1.

As described above, the therapeutic load is incorporated into the fast release implant by stirring homogenously into the molten PEG mixture. In some aspects, the therapeutic load includes Cis-Pt. The Cis-Pt may be incorporated into the molten PEG mixture at any suitable concentration including, but not limited to, 2 mg Cis-Pt/g PEG. In various other aspects, the Cis-Pt may be incorporated into the molten PEG mixture at a concentration of at least about 0.5 mg Cis-Pt/g PEG, at least about 1 mg Cis-Pt/g PEG, at least about 1.5 mg Cis-Pt/g PEG, at least about 2 mg Cis-Pt/g PEG, at least about 2.5 mg Cis-Pt/g PEG, at least about 3 mg Cis-Pt/g PEG, at least about 3.5 mg Cis-Pt/g PEG, at least about 4 mg Cis-Pt/g PEG, at least about 4.5 mg Cis-Pt/g PEG, at least about 5 mg Cis-Pt/g PEG, at least about 5.5 mg Cis-Pt/g PEG, at least about 6 mg Cis-Pt/g PEG, at least about 6.5 mg Cis-Pt/g PEG, at least about 7 mg Cis-Pt/g PEG, at least about 7.5 mg Cis-Pt/g PEG, at least about 8 mg Cis-Pt/g PEG, at least about 8.5 mg Cis-Pt/g PEG, at least about 9 mg Cis-Pt/g PEG, at least about 9.5 mg Cis-Pt/g PEG, or at least about 10 mg Cis-Pt/g PEG. In various additional aspects, the Cis-Pt may be incorporated into the molten PEG mixture at a concentration ranging from about 0.5 to about 1.5 mg Cis-Pt/g PEG, from about 1 to about 2 mg Cis-Pt/g PEG, from about 1.5 to about 2.5 mg Cis-Pt/g PEG, from about 2 to about 3 mg Cis-Pt/g PEG, from about 2.5 to about 3.5 mg Cis-Pt/g PEG, from about 3 to about 4 mg Cis-Pt/g PEG, from about 3.5 to about 4.5 mg Cis-Pt/g PEG, from about 4 to about 5 mg Cis-Pt/g PEG, from about 4.5 to about 5.5 mg Cis-Pt/g PEG, from about 5 to about 6 mg Cis-Pt/g PEG, from about 5.5 to about 6.5 mg Cis-Pt/g PEG, from about 6 to about 7 mg Cis-Pt/g PEG, from about 6.5 to about 7.5 mg Cis-Pt/g PEG, from about 7 to about 8 mg Cis-Pt/g PEG, from about 7.5 to about 8.5 mg Cis-Pt/g PEG, from about 8 to about 9 mg Cis-Pt/g PEG, from about 8.5 to about 9.5 mg Cis-Pt/g PEG, or from about 9 to about 10 mg Cis-Pt/g PEG.

In some aspects, the therapeutic load may further include additional compounds for evaluating the dissolution or distribution of the therapeutic load during treatment using the fast-release implant. Any suitable dye or contrast agent compatible with any suitable imaging modality used to assess dissolution or distribution may be included in the therapeutic load including, but not limited to, trypan blue and DiR, a near-infrared (NIR) dye.

In various aspects, the fast-release implants are configured to assume a solid phase at room temperature and to assume a liquid phase at the elevated temperature of the implant site within the cervical cancer lesion to facilitate the fast and local release of the therapeutic load. In various aspects, the fast-release implants may be administered by suitable means without limitation. In some aspects, the fast release implants may be administered without surgery using any suitable device including, but not limited to, a biopsy needle. In other aspects, the fast release implant may be administered surgically using any known surgical methods including, but not limited to catheter-based surgical methods.

In some aspects, the fast release implant may be used in a method for the treatment of a cervical cancer includes administering an effective dose of Cis-Pt in the form of the fast release implant described herein. In various other aspects, the method of treatment may further include administering the fast release implant in combination with a radiation treatment.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing cervical cancer in a subject in need of administration of a therapeutically effective amount of cisplatin (Cis-Pt), so as to kill and/or radiosensitize cervical cancer cells.

Methods described herein are generally performed on a subject in need thereof. A subject in need of the therapeutic methods described herein can be a subject having, diagnosed with, suspected of having, or at risk for developing cervical cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of Cis-Pt is, for example, an amount that would cause the desired therapeutic effect in a subject while minimizing undesired side effects. In various embodiments, an effective amount of Cis-Pt described herein can substantially inhibit the propagation of cervical cancer cells, slow the progress of cervical cancer, or limit the development of further cervical tumor growth or metastasis.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of Cis-Pt can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to kill or radiosensitize cervical cancer cells.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of Cis-Pt can occur as a single event or over a time course of treatment. For example, Cis-Pt can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cervical cancer.

The Cis-Pt fast release implant can be administered simultaneously or sequentially with another agent, such as an antibiotic, an anti-inflammatory, or another agent. For example, the Cis-Pt fast release implant can be administered simultaneously with another agent, such as an antibiotic or an anti-inflammatory. By way of another non-limiting example, the Cis-Pt fast release implant can be administered simultaneously with a radiation therapy suitable for the treatment of cervical cancer. Simultaneous administration can occur through the administration of separate compositions, each containing one or more of Cis-Pt, an antibiotic, an anti-inflammatory, or another agent. Cis-Pt can be administered sequentially with an antibiotic, an anti-inflammatory, or another agent. For example, Cis-Pt can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump that may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve the taste of the product; or improve the shelf life of the product.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the fast release implants described herein. Such packaging of the components separately can, if desired, be presented in a pack or dispenser device that may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrates, and/or may be supplied as an electronic-readable medium or video. Detailed instructions may not be physically associated with the kit; instead, a user may be directed to an Internet website specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: Localized Delivery of Cisplatin to Cervical Cancer

To develop an intra-cervical drug delivery system that allows cisplatin release directly into the tumor and minimize systemic side effects, the following experiments were conducted.

Twenty patient biopsies and five cell lines treated with cisplatin were analyzed for platinum content using inductively coupled plasma mass spectrometry. Polymeric implants loaded with cisplatin were developed and evaluated for degradation and drug release. The effects of local or systemic cisplatin delivery, in combination with radiation therapy, on drug biodistribution and tumor burden were evaluated in vivo.

Platinum levels in patient biopsies were 6-fold lower than the levels needed for efficacy and radiosensitization in vitro. Cisplatin local delivery implant remarkably improved drug specificity to the tumor and significantly decreased accumulation in the blood, kidney, and other distant normal organs, compared with traditional systemic delivery. The localized treatment further resulted in complete inhibition of tumor growth.

### Materials and Methods

### Chemicals and Reagents

Poly(ethylene glycol)-3350 (PEG3350) and poly(ethylene glycol)-400 (PEG400), cis-Diammineplatinum(II) dichloride (Cis-Pt), trypan blue, and nitric acid (HNO3) were purchased from Sigma Aldrich (St Louis, Mo.). The near-infrared (NIR) lipophilic tracer DiR was purchased from Invitrogen (Eugene, Oreg.). Corning Matrigel Basement Membrane Matrix Growth Factor Reduced (Corning, N.Y., N.Y.) was prepared following manufacturer instructions.

### Cell Culture

Human cervical cancer cell lines C33A, Me-180, HT3, Caski, and SiHa were purchased from American Type Culture Collection (ATCC; Manassas, Va.). Cell lines were cultured in Dulbecco's Modified Eagle Medium (DMEM; Gibco Life Technologies, Grand Island, N.Y.) supplemented with 10% fetal bovine serum (FBS; Gibco Life Technologies), 2 mmol/L of L-glutamine, 100 ng/mL penicillin, and 100 ng/mL streptomycin (Corning CellGro, Mediatech, Manassas, Va.). All cells were cultured at 37° C., 5% CO2 in a NuAire water jacket incubator (Plymouth, Minn.).

### Cervical Cancer Patient Biopsies

Patients with biopsy-proven cervical carcinoma were enrolled into an institutional review board-approved prospective study in which cervical tumor biopsies were obtained 3 weeks into chemoradiation treatment. All patients were treated with definitive chemoradiation treatment, which consisted of external beam radiation therapy to the pelvis (50.4 Gy) 4 days per week using intensity-modulated radiation therapy with a split-pelvis technique with 6 weekly brachytherapy boosts to a dose of 6.5 Gy per fraction to point A, starting at week 1. Concurrent with radiation therapy, all patients received the conventional treatment of cisplatin (40 mg/m², intravenous [IV]) weekly. Cervical tumor samples were collected immediately before delivery of the third brachytherapy treatment, approximately half-way through the treatment course. Samples were collected, immediately snap-frozen, and processed for storage. The average time between cisplatin administration and biopsy was 4.5 days (range, 1-17 days).

### Pt Accumulation in Cervical Cancer Human Biopsies

To determine tissue Cis-Pt accumulation within the patient biopsy samples, tissue digestion and inductively coupled plasma mass spectrometry (ICP-MS) were performed to quantify the levels of Pt in the samples. Briefly, biopsy samples were thawed and precisely weighed (±0.1 mg) before being digested in concentrated nitric acid (HNO₃) overnight. Samples were then diluted with deionized water to a final 5% HNO₃ (v/v) and digested using a microwave digestion system (MARS 6 Microwave Digestion System, CEM, Matthews, N.C.) at 200° C. for 45 minutes. Samples were analyzed with ICP-MS (ELAN DRC II ICP-MS, Perkin Elmer, Inc, Waltham, Mass.) for Pt content, against a calibration curve of Pt standards of 0, 0.1, 1, 10, 50, 100, and 250 parts per billion (ppb) in 5% HNO₃, which were prepared from 10 parts per million Pt standard solution (Inorganic Ventures, Christiansburg, Va.). Terbium was used as an internal standard throughout the ICP-MS analysis. The average Pt content in clinical biopsies (Av-Pt-Clin) was calculated.

### In Vitro Efficacy of Cis-Pt

The in vitro cytotoxicity on cervical cancer cell lines was assessed using an MTT assay (Sigma Aldrich). Briefly, cells were seeded at 5×10³ cells/well in 96-well plates overnight at 37° C. to promote their adhesion to the plates. The cells were treated with increasing concentrations of Cis-Pt of 1, 2.5, 5, and 10 μM. After 48 hours of treatment, MTT solution was added to the cells for 3 hours, and then the stop solution was added to dissolve the formazan crystals overnight. The wells were read with SpectraMax i3 multimode microplate spectrophotometer (Molecular Devices, San Jose, Calif.) at 570 nm. Dose-response curves were graphed and the average dose-response curve was calculated. The average half-maximal inhibitory concentration (Av-IC₅₀) of Cis-Pt from all 5 cell lines was also calculated.

### Pt Content in Cell Lines In Vitro

Cervical cancer cell lines (10×10⁶ cells) were treated Cis-Pt at Av-IC₅₀ for 48 hours. Cells were then lifted with trypsin (0.05% Trypsin-EDTA, Gibco Life Technologies), spun down, and washed 3 times, to eliminate free Cis-Pt. Cell pellets were then weighed, digested, and analyzed by ICP-MS, and the average Pt content in cell lines in vitro (Av-Pt-in vitro) was calculated.

In Vitro Radio-Sensitization with Cis-Pt

Clinically relevant Pt concentration (Clin-conc) was estimated as a fraction of Av-IC₅₀ based on the relative difference between tissue Pt contents in clinical biopsies and in vitro: Av-IC₅₀×(Av-Pt-Clin/Av-Pt-in vitro). The radiosensitizing effect of Cis-Pt on cervical cancer cell lines was assessed using an MTT assay. Briefly, cells were treated with or without Cis-Pt at Av-IC₅₀ (6 μM) or Clin-conc (1 μM) for a duration of 24 or 48 hours. Additionally, cells were also treated with or without a single-fraction dose of 6 Gy (4 Gy/min) using an RS2000 160 kV x-ray Irradiator equipped with a 0.3-mm copper filter (Rad Source Technologies Inc, Buford, Ga.), at the beginning of the treatment. At the end of each treatment, an MTT assay was performed to determine the survival of the cells.

The combination effect between Cis-Pt treatment and radiation was evaluated by calculating the combination index (CI) using the formula: CI=(Ea×Eb)/Eab, where Ea and Eb are the individual effects (fractional killing, 0≤Ei≤1) and Eab is the combination effect of drugs a and b. This index represents the ratio between the predicted combination effect and the actual combination effect. When the CI=1, the effect is additive. When the CI<1, the effect is supra-additive; when CI>1, the effect is sub-additive.

### In Vitro Clonogenic Survival

C33A cervical cancer cells (0.5×10⁶ cells/condition) were plated in T25 flasks 24 hours before treatment. Cells were treated with Cis-Pt at 0, 1, 3, or 6 μM for 4 hours, and subjected to irradiation at 0 or 6 Gy (4 Gy/min). Cells were then lifted with trypsin, counted, and were plated in triplicates for each condition at 500 cells per well in 24 well plates. After 12 days of culture, cells were fixed and stained with 0.5% Crystal Violet (Sigma Aldrich) in methanol, washed, and dried. The number of clones that resulted in each well was analyzed by ImageJ software (NIH, Bethesda, Md.).

### PEG-Implant Preparation

PEG implants were prepared from PEG3350 and PEG400 mixtures with mass ratios of 80:20. The PEG implants were loaded with trypan blue (for in vitro dissolution studies), near-infrared (NIR) dye DiR (for in vivo dissolution studies), or Cis-Pt (for in vitro release, in vivo biodistribution, and efficacy studies). The different load compounds were homogenously stirred into the molten polymers mixture before cooling.

Cis-Pt PEG implants for in vivo biodistribution and efficacy studies were prepared at 2 mg Cis-Pt/g PEG and allowed to cool in molds. Implants for in vivo studies were obtained by biopsy aspiration needles (BD, Vernon Hills, Ill.) and weighed approximately 10±0.5 mg. Each implant contained about 0.02 mg of Cis-Pt. Administration of each implant was equivalent to a dose of 1 mg/kg in a 20 g animal.

### In Vitro Dissolution and Drug Release of PEG Implant

Trypan blue implants were placed into a 6-well plate and allowed to dissolve in PBS (3 mL/well) over a shaker at 37° C. Dissolution rate of the implant was monitored by photographs taken at multiple time points (0-30 minutes) until disappearance. The size of the remaining implant at each time point was analyzed using ImageJ software.

To evaluate the release profile of Cis-Pt from implants in vitro, Cis-Pt (10 mg/g PEG) was loaded to the implants as described above. Implants were placed into a 6-well plate PBS (3 mL/well) over a shaker at 37° C. Release rate was monitored by sampling 100 μL of buffer at different time points (0-30 min) and replacing it with fresh buffer. The amount of released Pt was measured by ICP-MS. The release was calculated as: (Amount at time point−Blank)/(Total amount−Blank)×100%.

### In Vivo Dissolution Rate

Balb/c mice, female, 6 to 8 weeks old (Charles Rivers Laboratories, Wilmington, Md.), were anesthetized with ketamine/xylazine. To evaluate the Cis-Pt release profile from the implants in vivo, a small incision was made to the abdominal wall and a DiR-loaded implant was inserted into the peritoneal cavity of each mouse (n=3). Release rate was monitored by imaging the infrared signal in each mouse at different time points (0-30 min) using Pearl NIR fluorescent imager (LI-COR Biotechnology, Lincoln, Nebr.). The in vivo release was calculated as: (Area with infrared)/(Area of the whole abdomen)×100%.

### In Vivo Biodistribution

C33A cells (2×10⁶/mouse) were suspended in 50% Matrigel and 50% DMEM media. The suspended cells were injected subcutaneously around the lower back of 10 athymic nude mice, female, 6 to 8 weeks old (Charles River Laboratories). Tumor progression was confirmed after 3 weeks as tumor volume of >200 mm³, measured by caliper and calculated using the formula: V=0.5ab², in which a and b are the major and minor axis of the tumor, respectively. Mice were randomized into 2 groups (n=5) and treated with either a tail vein injection of free Cis-Pt at the dose of 1 mg/kg (Cis-Pt-IV) or intratumoral implantation of Cis-Pt-loaded implant at the dose of 1 mg Cis-Pt/kg (Cis-Pt-Implant).

To mimic the anatomic structure of the cervix for local delivery, an os-like cavity was developed in the center of the subcutaneous tumor by taking out a biopsy from the center of the tumor using a Jamshidi bone marrow biopsy aspiration needle (BD, Vernon Hills, Ill.). For the local delivery, implants were inserted into the tumor cavity by a biopsy needle. The cavity was then closed using VetClose surgical glue (Henry Schein, Dublin, Ohio). Mice were sacrificed 24 hours post-treatment and various organs (tumor, heart, kidney, liver, spleen, and blood) were harvested, weighed, digested with nitric acid/microwave, and analyzed for Pt content using ICP-MS. The concentration of Pt in each tissue was determined as: Pt Amount/Tissue Weight.

The selected mouse model incorporated useful features of multiple mouse models to assess the effects of localized delivery of the payload from the PEG implant on cervical cancer. Although orthotopic models represent the biological and anatomic characteristics of cervical cancer, their use in the context of intracervical drug delivery is a challenge owing to the small size of the mouse cervix and the corresponding technical challenges in developing implants of this size. Alternatively, subcutaneous models provide the proper size of the tumor but lack the anatomic structure needed for intratumoral implantation. As described above, the modification of the subcutaneous tumors in the mouse model as described above provided a tumor representative of the biological and anatomic characteristics of cervical cancer with sufficient room to receive the PEG implant.

### In Vivo Tumor Penetration

Subcutaneous cervical tumors were inoculated in 6 athymic nude mice as described above. Mice were treated with either an intratumoral empty implant (n=3) or an intratumoral Cis-Pt-loaded implant (n=3). After 24 hours, mice were sacrificed and the tumors were resected. The tumors were fixed with PFA and separated into halves for ICP-MS elemental analysis and immunofluorescence analysis, respectively.

For ICP-MS, tumors were washed with PBS and cut into inner, middle, and outer layers, corresponding to the distance from the drug implant (approximately 1 mm each layer). Each layer was digested and analyzed as described above. Pt levels were represented as ng Pt/g tissue and adjusted for empty implant background signal.

For immunofluorescence, tumors were frozen in OCT compound (Fisher Scientific, Pittsburgh, Pa.), and sectioned to 10-μM slices using Leica CM1950 Cryostat (Leica, Wetzlar, Germany). Cryosections were blocked with 10% FBS, stained with 1:200 dilution of Cisplatin-modified DNA antibody (CP9/19) (Novus Biologicals, Centennial, Colo.) for 1 hour at room temperature, washed with PBS, followed by staining with 1:1000 dilution of goat anti-Rat IgG (H+L) Cross-Adsorbed Secondary Antibody, Alexa Fluor 488 (Invitrogen, Carlsbad, Calif.) for 30 minutes at room temperature, and washed with PBS. Sections were counterstained with ProLong Gold Antifade Mountant with DAPI (Invitrogen) and analyzed by AxioPlan 2 Fluorescence Microscope (Carl Zeiss, Oberkochen, Germany).

### In Vivo Efficacy

C33A cells (2×10⁶/mouse) were suspended in 50% Matrigel and 50% DMEM media. The suspended C33A cells were injected subcutaneously around the lower back of 42 female athymic nude mice that were 6 to 8 weeks old. Tumor progression was confirmed after 3 weeks, and mice were then randomized into 6 groups (n=7) and treated with: (1) tail vein injection of PBS (Vehicle-IV), (2) Cis-Pt-IV at 1 mg/kg, (3) Cis-Pt-Implant at 1 mg Cis-Pt/kg, (4) Vehicle-IV+radiation (5 days×2 Gy/day), (5) Cis-Pt-IV+radiation, and (6) Cis-Pt-Implant+radiation.

In a separate experiment, the effect of a fractionated radiation dose was compared to a single high dose of radiation administered concurrently with Cis-Pt-Implants. C33A cells (2×106/mouse) were suspended in 50% Matrigel and 50% DMEM media. The suspended C33A cells were injected subcutaneously around the lower back of 14 female athymic nude mice that were 6 to 8 weeks old (Charles Rivers Laboratories). Tumor progression was confirmed after 3 weeks, mice were then intratumorally implanted with Cis-Pt-loaded implant (1 mg/kg) and randomized into 2 groups (n=7), treated with: (1) fractionated radiation (5 days×2 Gy/d), mimicking external beam radiation; or (2) a single high dose of 8 Gy, mimicking brachytherapy.

For the in vivo experiments above, tumor progression was monitored by measurement of tumor sizes in all treatment groups every 2 days. Radiation treatment for mice was 2 Gy/d for 5 consecutive days, or a single high dose of 8 Gy, using an RS2000 160 kV X-ray Irradiator equipped with a 0.3-mm copper filter (Rad Source Technologies Inc, Buford, Ga.), starting immediately after the first vehicle or Cis-Pt treatment. The mice were protected with lead shielding such that only the subcutaneous tumors were exposed to radiation.

### Statistical Analysis

All experiments were performed in at least triplicate, and all cell line experiments were repeated at least 3 times. Results were expressed as means±standard deviation, and statistical significance was analyzed using Student t-test and ANOVA. P values less than 0.05 were used to indicate statistically significant differences.

### Results

### Effect of Cis-Pt as a Radio-Sensitizer in Cervical Cancer

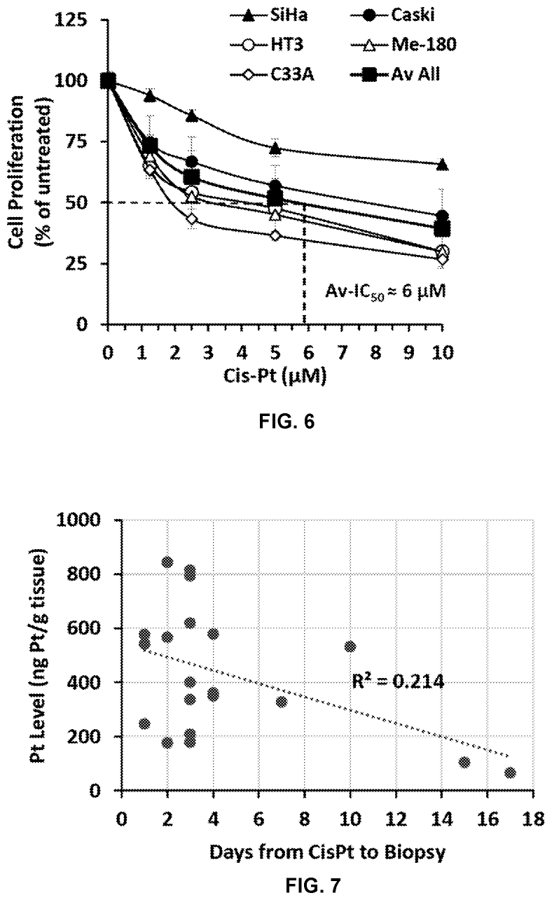

The cytotoxicity profiles of Cis-Pt in human cervical cancer were established by creating dose-response curves for 5 cervical cancer cell lines (C33A, Me-180, HT3, Caski, and SiHa). The average dose-response curve was determined and the average IC₅₀ (Av-IC₅₀) was determined using this curve (FIG. 6). The Av-IC₅₀ was found to be 6 μM and represents the in vitro efficacious concentration.

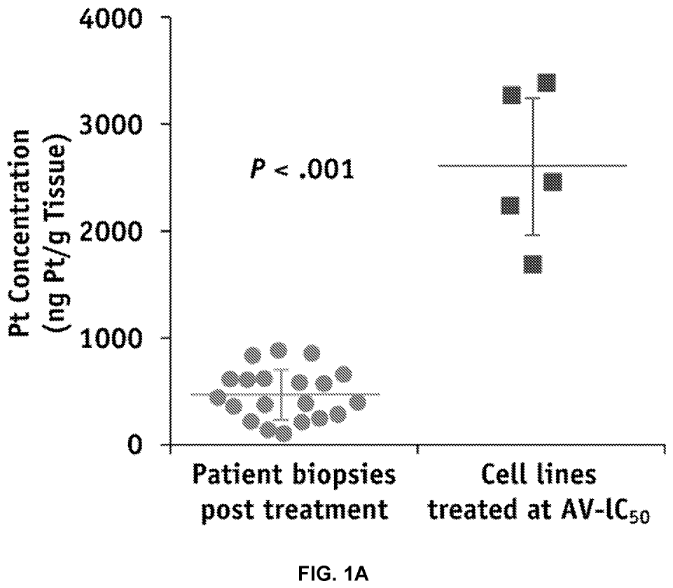

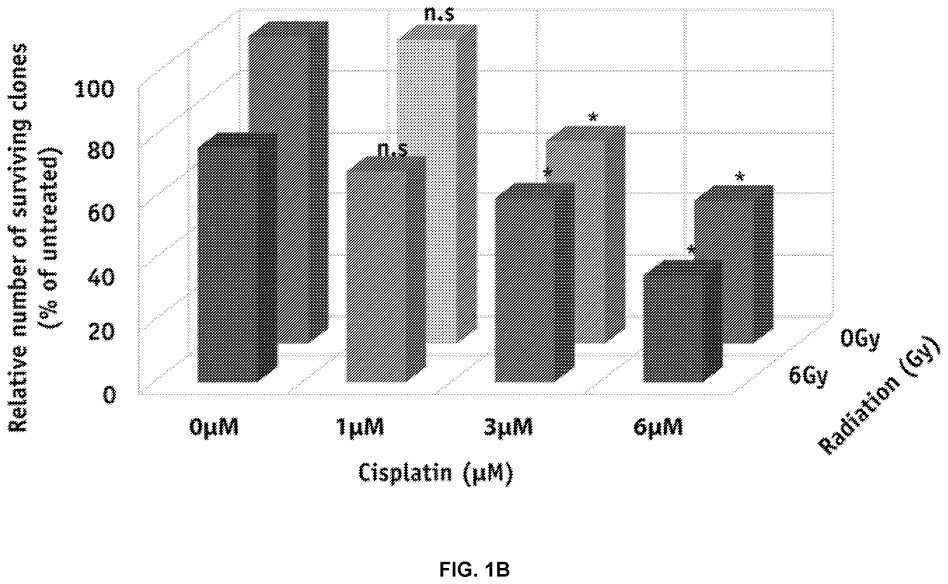

The Pt content in 20 tumor biopsies from cervical cancer patients approximately 3 weeks into definitive treatment with chemoradiation was determined and compared to the Pt content found in cell lines treated with Cis-Pt at AV-IC₅₀. Patient characteristics are presented in Table 1 below. Pt content in biopsies was represented as a function of time since the last cisplatin injection, showing that the time of biopsy was not a major factor for the level of Pt within the tumor (FIG. 7). Pt levels in cell lines treated at in vitro efficacious concentration of 6 μM averaged to about 2560 ng Pt/g tissue, while Pt levels detected in patient tissues averaged to about 430 ng Pt/g tissue (FIG. 1A). Collectively, these results demonstrated that drug accumulation in the patient tissues was about 6-fold lower, which was equivalent to the result of a 1-μM treatment condition. According to the dose-response curves found previously, this clinically relevant concentration of 1 μM would have limited cytotoxicity.

TABLE 1
Clinical characteristics of patients with cervical tumor biopsies
Clinical Characteristic, All Patients (N = 20)

| Median age | 53.7 y (range, 33-76) | |
|---|---|---|
| FIGO stage | Value (%) | |
| IB1 | 1 | (5) |
| IB2 | 5 | (25) |
| IIB | 3 | (15) |
| IIIA | 1 | (5) |
| IIIB | 9 | (45) |
| IVB | 1 | (5) |
| Histology | | |
| Squamous | 17 | (85) |
| Adenocarcinoma | 3 | (15) |

FIGO = International Federation of Gynecology and Obstetrics, 2009, staging.

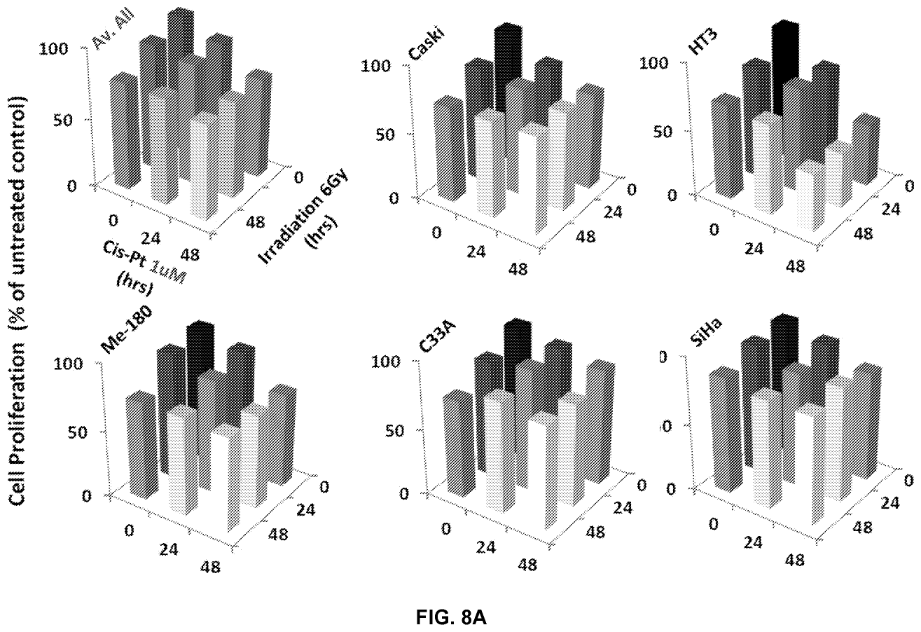

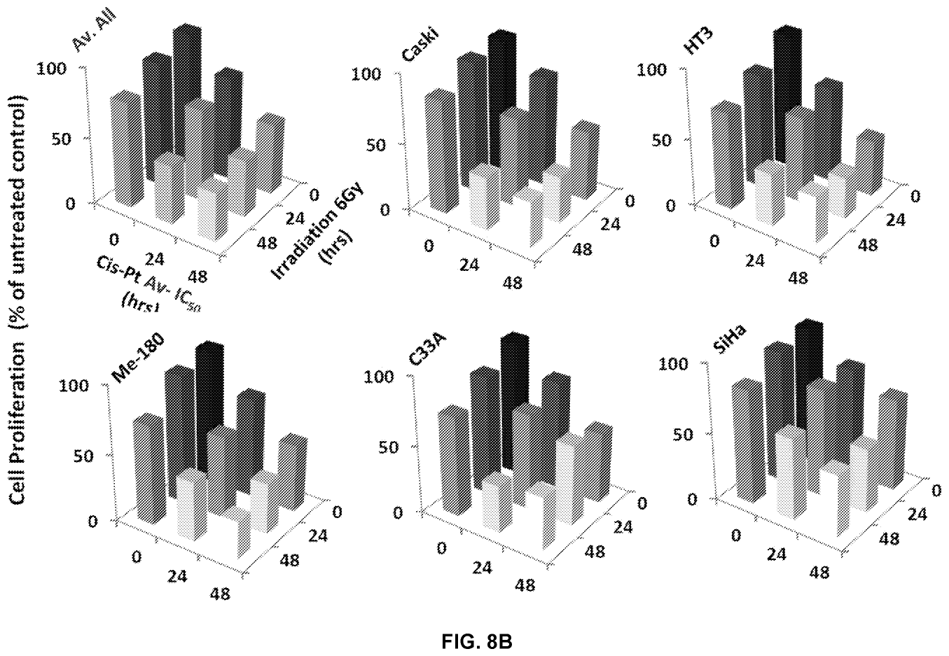

How low Cis-Pt accumulation in cervical cancer tissues would affect Cis-Pt as a radio-sensitizer was also tested. Cell lines were treated with Cis-Pt at the in vitro efficacious concentration of 6 μM or at the clinically relevant concentration of 1 μM, with or without concurrent radiation. 6 μM Cis-Pt was able to induce significant sensitization to radiation therapy in all cell lines (FIG. 8A); on the other hand, 1 μM Cis-Pt induced limited radio-sensitization in the cell lines (FIG. 8B). Furthermore, the combination index (CI) was calculated to compare the predicted combination effect and the actual combination effect of Cis-Pt and radiation in both cases. Specifically, CI for 6 μM treatment showed a supra-additive effect, whereas CI for 1 μM treatment showed sub-additive effect (Table 2).

TABLE 2
Effect of Combined Cis-Pt and Radiation Treatments

| | | Cis-Pt (6 μM) | | Cis-Pt (1 μM) | |
|---|---|---|---|---|---|
| Combination Index (CI) | | 24 hrs. | 48 hrs. | 24 hrs. | 48 hrs. |
| Radiation (6 Gy) | 24 hrs. | 0.9675 | 0.8279 | 1.0563 | 1.0555 |
| | 48 hrs. | 0.7328 | 0.8927 | 1.1070 | 1.2264 |

Drug Interactions: CI < 1: Supra-additive; CI = 1: Additive: CI > 1: Sub-Additive

To test how the discrepancy in Cis-Pt levels affected the clonogenic ability in cervical cancer cells, C33A cell lines were treated with 0, 1, 3, or 6 μM Cis-Pt, with or without concurrent radiation. Analyzing the relative number of surviving clones revealed that 1 μM concentration was unable to limit clonogenic cell survival of cervical cancer cell lines, neither alone nor in combination with radiation. In contrast, higher doses (3 and 6 μM) significantly restricted clonogenic growth (FIG. 1B).

The synergistic interaction between Cis-Pt and irradiation can be achieved with higher Pt accumulations in tumor tissues, which can potentially be accomplished with localized delivery of Cis-Pt into the tumor, before radiation therapy.

### Development of Localized Fast Release Delivery System for Cis-Pt

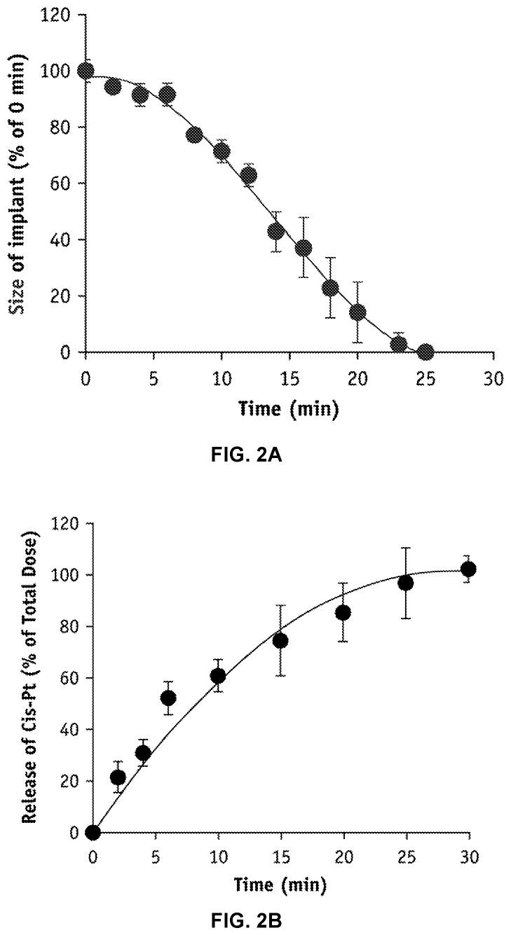

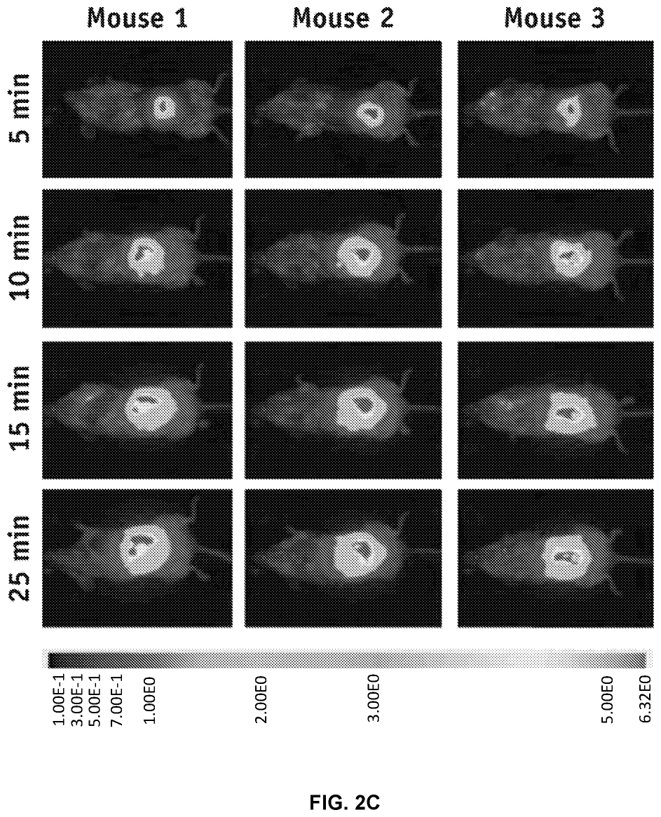

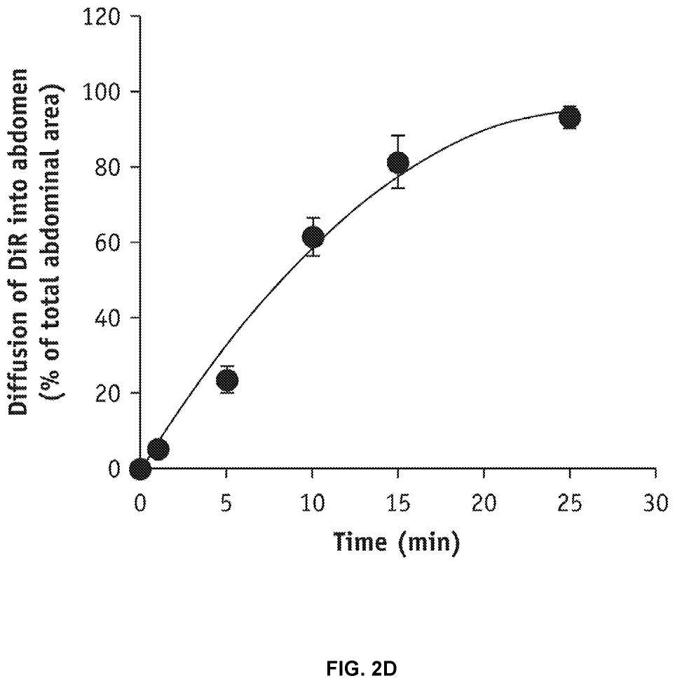

A combination of PEG with low and high molecular weights, PEG400 and PEG3350, was used for the development of a fast release delivery implant. The degradation of the implant in vitro was characterized as described above and found to degrade gradually within 25 minutes (FIG. 2A). Similarly, Cis-Pt-loaded implants released their content gradually into the buffer and Pt content reached a plateau around 25 minutes (FIG. 2B).

Comparable results were also observed in vivo. DiR-loaded implants inserted into the peritoneal cavity in mice degraded and released dye within 25 minutes, as shown in representative NiR images (FIG. 2C) and quantitative measurements (FIG. 2D).

In Vivo Biodistribution of Cis-Pt after Localized or Systemic Administration

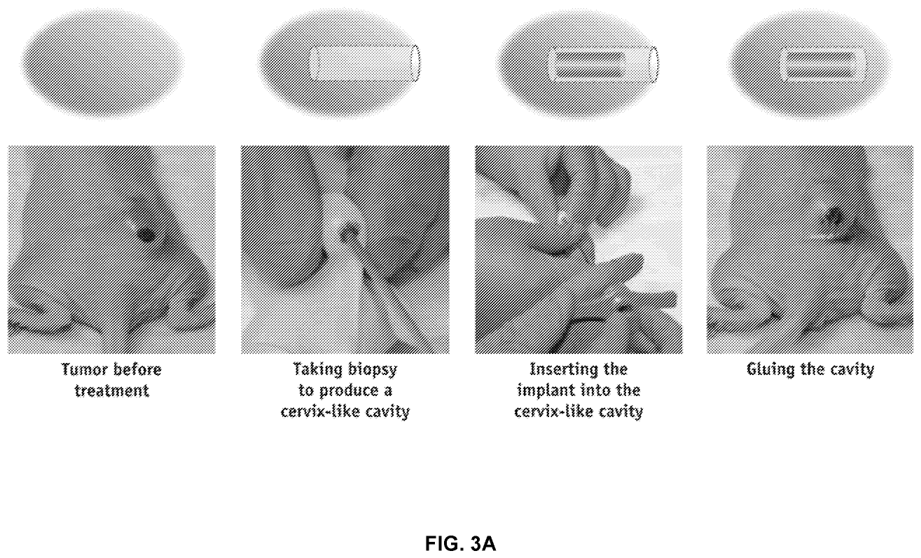

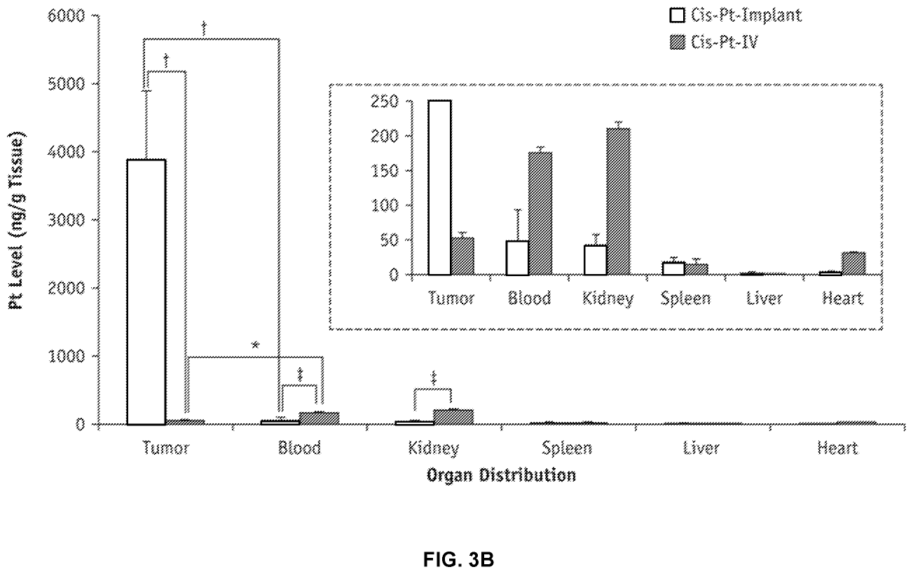

Patients with cervical cancer undergo brachytherapy treatment as a standard part of care. During each of the treatments, a device is implanted through the cervix tumor and into the uterus, presenting an opportunity to introduce local drug delivery in conjunction with this implant. To this end, a subcutaneous model of cervical cancer tumor was developed in which a cavity was created in the center of the tumor to mimic the anatomic structure of the human cervix, allowing insertion of candidate drug delivery devices (FIG. 3A). Mice were administered Cis-Pt with either systemic Cis-Pt (Cis-Pt-IV) according to clinical practice, or with the localized Cis-Pt implants (Cis-Pt-Implant) described above. The biodistribution profiles for the 2 treatment groups were compared by measuring the Pt amount in various organs 24 hours after IV or local implantation. Pt accumulated in the tumor at 73.4-fold higher concentrations after local (implant) delivery (3896 ng Pt/g tissue) compared with systemic (injection) delivery (53 ng Pt/g tissue). Pt levels detected in the blood and kidney were significantly lower for local Pt delivery by implant compared with systemic Pt delivery by injection (FIGS. 3B and 3C). Tumor-to-blood ratios of Pt were calculated to be 80.3 for localized delivery and 0.3 for systemic delivery, as summarized in Table 3 below.

TABLE 3
Biodistribution concentrations of Cis-Pt administered by implant and IV.
Pt Concentration Ratio

| | | |
|---|---|---|
| Cis-Pt-Implant/Cis-Pt-IV | Tumor | 73.47 |
| | Blood | 0.27 |
| | Kidney | 0.19 |
| Tumor/Blood | Cis-Pt-Implant | 80.32 |
| | Cis-Pt-IV | 0.30 |

### In Vivo Tumor Penetration of Cis Pt Implants

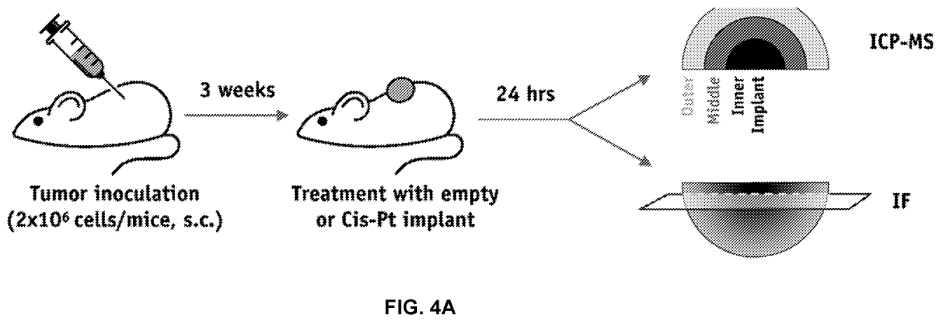

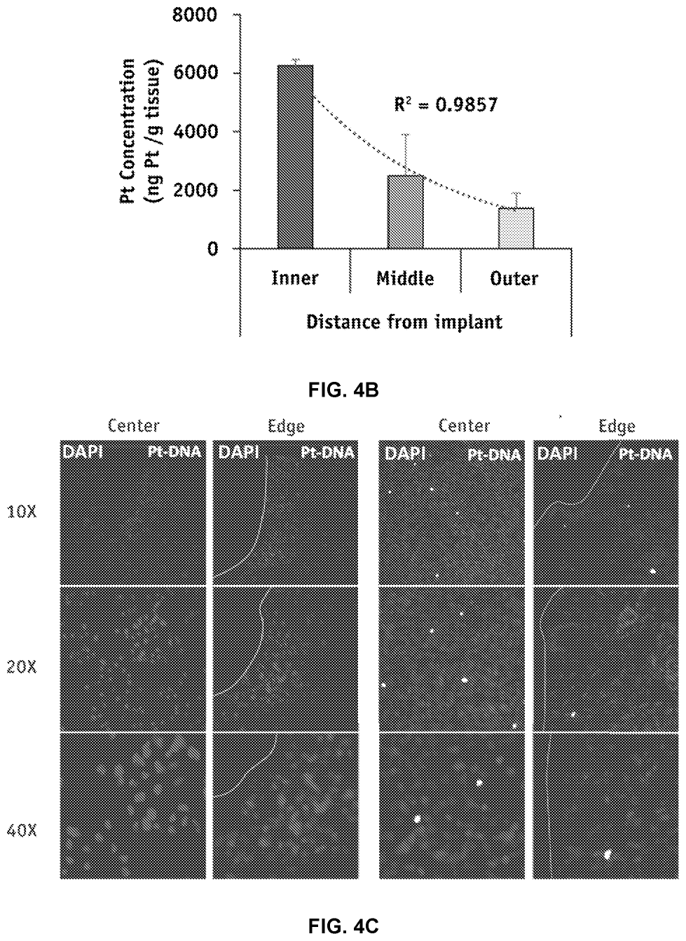

To determine how far Cis-Pt would be able to diffuse from the implants into the depth of the tumor, Pt distribution in the tumors was assessed using 2 different methods: quantitatively using ICP-MS, and qualitatively using immunofluorescence (FIG. 4A). ICP-MS analysis revealed Cis-Pt penetrates all layers of the tumors. The levels of Cis-Pt were inversely correlated with the distance from the implant; the tumor layers closest to the implant had high concentrations of Pt, and outer layers had moderate levels of Pt, all of which are higher than what we detected in tumor biopsies from patients (FIG. 4B). For immunofluorescence, tumor sections were stained for Cis-Pt-DNA adducts, and regions at the center or the edge of the stained tumor sections were imaged. Cis-Pt-DNA adducts were abundant in the central regions and to a lower extent in the edges of the tumors (FIG. 4C).

### In Vivo Efficacy of Localized Cis-Pt Delivery

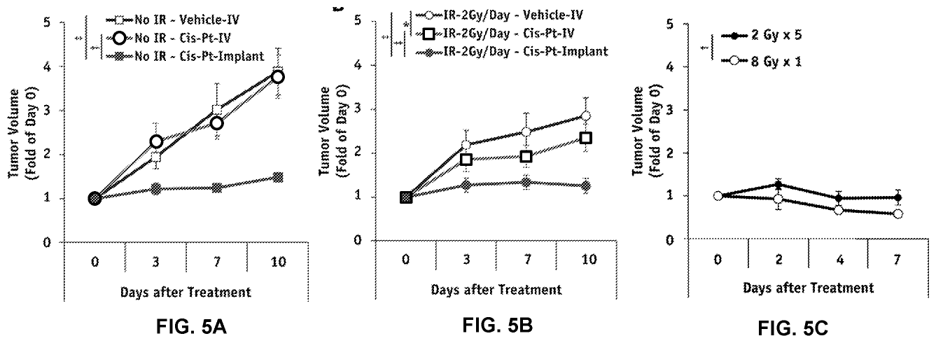

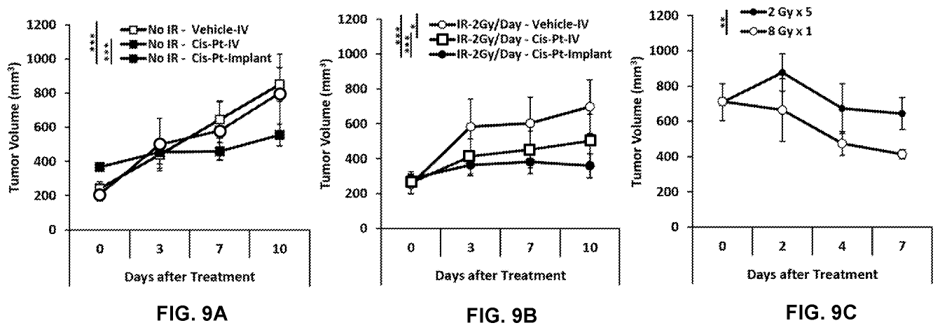

The effect of Cis-Pt on cervical tumor progression when delivered locally or systemically, with or without concurrent radiation was compared. Systemic delivery of Cis-Pt did not alter tumor progression of cervical tumors in vivo, and localized delivery of the same dose resulted in complete inhibition of tumor growth (FIGS. 5A and 9A). Furthermore, when Cis-Pt treatments were combined with fractionated radiation (2 Gy×5 days, representing clinical dose), systemic Cis-Pt delivery improved treatment outcome compared with radiation alone, and localized Cis-Pt treatment profoundly improved the effect of radiation in vivo (FIGS. 5B and 9B), implying augmented radio-sensitization.

The outcomes of treatments combining the localized Cis-Pt implant with either a single high dose of radiation (8 Gy; similar to the brachytherapy boost portion of standard-of-care radiation therapy for cervix) or 5 consecutive fractionated radiation doses (2 Gy/d; representing conventionally fractionated external beam irradiation) were compared. Localized Cis-Pt implant showed better results when combined with the single high dose compared with combination with fractionated therapy (FIGS. 5C and 9C), suggesting that administration of localized Cis-Pt immediately before brachytherapy may result in considerable clinical benefit.

The results of these experiments demonstrated the development and use of a polymeric delivery implant for fast and local Cis-Pt delivery to cervical cancer. The Cis-Pt implant demonstrated an outstanding biodistribution profile with superior tumor drug accumulation and significantly lower drug accumulation in normal tissues compared with systemic delivery. The Cis-Pt implant also exhibited in vivo antitumor therapeutic efficacy.

What is claimed is:

1. A fast release implant for the treatment of a cervical cancer, the implant comprising a polymer and a therapeutic load homogeneously distributed throughout the polymer, wherein the implant assumes a solid phase at room temperature and assumes a liquid phase at a body temperature of a patient.

2. The implant of claim 1, wherein the polymer comprises a mixture of a high MW PEG and a low MW PEG.

3. The implant of claim 2, wherein the high MW PEG is PEG3350 and the low MW PEG is PEG400.

4. The implant of claim 2, wherein the implant comprises a molar ratio of high MW PEG:low MW PEG of 80:20.

5. The implant of claim 1, wherein the therapeutic load comprises Cis-Pt.

6. The implant of claim 5, wherein the therapeutic load comprises Cis-Pt at a concentration of about 2 mg Cis-Pt/g PEG.

7. The implant of claim 1, wherein the therapeutic load further comprises a dye or a contrast agent.

8. The implant of claim 7, wherein the dye or the contrast agent is selected from the group consisting of trypan blue, DiR, and any combination thereof.

9. A method of treating cervical cancer within a patient in need, the method comprising implanting a fast release implant containing a therapeutic load within a cervical cancer lesion, the implant comprising a polymer and the therapeutic load homogenously distributed throughout the polymer, wherein the implant assumes a solid phase at room temperature and assumes a liquid phase at a body temperature of a patient.

10. The method of claim 9, wherein the polymer comprises a mixture of a high MW PEG and a low MW PEG.

11. The method of claim 10, wherein the high MW PEG is PEG3350 and the low MW PEG is PEG400.

12. The method of claim 10, wherein the implant comprises a molar ratio of high MW PEG:low MW PEG of 80:20.

13. The method of claim 9, wherein the therapeutic load comprises a therapeutically effective amount of Cis-Pt.

14. The method of claim 13, wherein the therapeutic load comprises Cis-Pt at a concentration of about 2 mg Cis-Pt/g PEG.

15. The method of claim 9, wherein the therapeutic load further comprises a dye or a contrast agent.

16. The method of claim 15, wherein the dye or the contrast agent is selected from the group consisting of trypan blue, DiR, and any combination thereof.
